(19) United States
(12) Patent Application Publication
(10) Pub. No.: US 2019/0153013 A1
(43) Pub. Date: May 23, 2019

(54) METHOD FOR EXTRACTING ACTIVE INGREDIENTS

(71) Applicant: TCI CO., LTD, Taipei (TW)

(72) Inventors: Yung-Hsiang LIN, Taipei (TW); Chien-Yu LU, Taipei (TW)

(21) Appl. No.: 16/198,454

(22) Filed: Nov. 21, 2018

Related U.S. Application Data
(60) Provisional application No. 62/590,125, filed on Nov. 22, 2017.

(51) Int. Cl. C07H 1/08; C07D 211/82; C07D 309/10; C07D 311/76; C07D 209/44; C07H 17/02; B01D 11/02

(52) U.S. Cl. CPC C07H 1/08; C07D 211/82; C07D 309/10; B01D 11/0257; C07D 209/44; C07H 17/02; C07D 311/76

(57) ABSTRACT

A method for extracting active ingredient(s) from a raw material to be extracted is provided. The method comprises the following steps: (1) mixing a raw material to be extracted with an aqueous solvent to provide a mixture; (2) conducting a homogenization to the mixture to obtain a first extract with active ingredient(s); and (3) conducting a high pressure homogenization to the first extract to obtain a second extract with active ingredient(s), wherein the homogenization in step (2) is conducted at a temperature ranging from normal temperature to 100° C.

### FIELD OF THE INVENTION

The present invention relates to a method for extracting an active ingredient from a raw material to be extracted, and particularly relates to a method for extracting an active ingredient from a raw material to be extracted by using a high pressure homogenization.

### BACKGROUND OF THE INVENTION

It is known that plants contain many active ingredients, which are beneficial for the human body, such as anthocyanidin in black tomatoes, serotonin in banana peels, and betalain, betaxanthin, polyphenol and flavonoid in Chenopodium formosanum, etc.

It is known that the active ingredients in plants can be extracted by way of such as the dipping method, expeller process, solvent extraction method, ultrasonic extraction method or supercritical extraction method. However, conventional methods are time-consuming, inefficient in extraction, and unable to be used in mass-production, and thus should be improved.

### SUMMARY OF THE INVENTION

In view of the deficiencies of the conventional extraction techniques, the present invention provides a method for extracting an active ingredient from a raw material to be extracted, and the method can extract the active ingredient from the raw material in a large amount.

Therefore, an objective of the present invention is to provide a method for extracting an active ingredient from a raw material to be extracted, comprising the following steps:
(1) mixing the raw material to be extracted with an aqueous solvent to provide a mixture;
(2) conducting a homogenization of the mixture to obtain a first extract with the active ingredient; and
(3) conducting a high pressure homogenization of the first extract to obtain a second extract with the active ingredient,
wherein the homogenization in step (2) is conducted at a temperature ranging from normal temperature to 100° C.

In some embodiments of the present invention, the high pressure homogenization in step (3) is conducted at a pressure ranging from 100 bar to 2,000 bar.

In some embodiments of the present invention, the homogenization in step (2) is conducted at a temperature ranging from normal temperature to 90° C.

In some embodiments of the present invention, the raw material to be extracted is selected from the group consisting of Chenopodium formosanum, black tomato, banana peel and combinations thereof.

In some embodiments of the present invention, the raw material to be extracted is Chenopodium formosanum, and the active ingredient is selected from the group consisting of betalain, betaxanthin, polyphenol, flavonoid and combinations thereof. Preferably, the homogenization in step (2) is conducted at a normal temperature or a temperature ranging from 80° C. to 90° C.

In some embodiments of the present invention, the raw material to be extracted is a black tomato, and the active ingredient is anthocyanidin. Preferably, the homogenization in step (2) is conducted at a temperature ranging from 35° C. to 45° C.

In some embodiments of the present invention, the raw material to be extracted is a banana peel, and the active ingredient is serotonin. Preferably, the homogenization in step (2) is conducted at a temperature ranging from 80° C. to 90° C.

In some embodiments of the present invention, the method further comprises, before step (1), breaking the raw material to be extracted.

In some embodiments of the present invention, the method further comprises, before step (3), sieving the homogenate by using a sieve with 20 mesh to 400 mesh.

In some embodiments of the present invention, the aqueous solvent is water.

To render the above objectives, technical features, and advantages of the present invention more apparent, the present invention will be described in detail with reference to some embodiments hereinafter.

### DESCRIPTION OF THE PREFERRED EMBODIMENT

The following paragraphs will describe some of the embodiments of the present invention in detail. However, without departing from the spirit of the present invention, the present invention may be embodied in various embodiments and should not be limited to the specific embodiments described in the specification.

Unless it is additionally explained, the expression “a,” “an,” “the,” or the like recited in the specification (especially in the claims) should include both the singular and plural forms.

As used herein, the term “about” in quantitative terms refers to plus or minus an amount that is general and reasonable to persons skilled in the art. In addition, the numerical ranges (e.g., 5 to 100) used in this specification should be construed as including all of the rational numbers in the ranges and ranges consisting of any rational numbers in the ranges. Therefore, the numerical ranges used in this specification should include all the possible combinations of numerical values between the lowest value and the highest value listed therein.

As used herein, the terms such as “first”, “second”, or the like are used to distinguish different components, not terms supplying a numerical limit.

The present invention provides a method for extracting an active ingredient from a raw material to be extracted, comprising the following steps: (1) mixing the raw material to be extracted with an aqueous solvent to provide a mixture; (2) conducting a homogenization of the mixture to obtain a first extract with the active ingredient; and (3) conducting a high pressure homogenization of the first extract to obtain a second extract with the active ingredient. Surprisingly, by the synergistic effects achieved by the combination of step (1), step (2) and step (3), the method of the present invention can extract a much larger amount of the active ingredient from the raw material to be extracted.

In the method of the present invention, the mixing process of step (1) can be conducted at a normal temperature, wherein the raw material to be extracted is preferably subjected to a breaking process to increase the area of contact of the raw material to be extracted with the aqueous solvent, thereby enhancing the efficiency of the subsequent extraction. The breaking process can be conducted by any conventional methods by using, such as scissors, crushers, grinders, etc.

The raw material to be extracted can be any known plant which contains an active ingredient beneficial to human body. In some embodiments of the present invention, the raw material to be extracted is a black tomato, banana peel (e.g., peels of Formosana, Pei Chiao, Red Dacca, oolong banana or Tai-Chiao no. 5) or Chenopodium formosanum (e.g., shelled Chenopodium formosanum).

The aqueous solvent can be a known solvent that is able to extract an active ingredient from the raw material to be extracted. The amount of the aqueous solvent is not particularly limited. Example of the aqueous solvent includes but is not limited to water.

In the method of the present invention, the homogenization in step (2) is conducted at a temperature ranging from normal temperature to 100° C., and preferably from normal temperature to 90° C., such as 25° C., 30° C., 35° C., 40° C., 45° C., 50° C., 55° C., 60° C., 70° C., 75° C., 80° C. or 85° C. The homogenization can primarily homogenize the raw material to be extracted, uniformly disperse it in the aqueous solvent and release the active ingredient(s), thereby obtaining a first extract with the active ingredient(s). When the raw material to be extracted is Chenopodium formosanum, the homogenization in step (2) is conducted at a normal temperature or a temperature ranging from 80° C. to 90° C. Under the condition of temperature as described, a larger amount of active ingredient can be extracted, including betalain, betaxanthin, polyphenol and flavonoid. When the raw material to be extracted is a black tomato, the homogenization in step (2) is conducted at a temperature ranging from 35° C. to 45° C. Under the condition of temperature as described, a larger amount of active ingredient, such as anthocyanidin, can be extracted. When the raw material to be extracted is a banana peel, the homogenization of step (2) is conducted at a temperature ranging from 80° C. to 90° C. Under the condition of temperature as described, a large amount of active ingredient, such as serotonin, can be extracted.

Optionally, the homogenization in step (2) can be conducted by multiple stages. For example, to enhance the efficiency of homogenization and reduce the consumption of the homogenization apparatus, when the raw material to be extracted is hard and big, the raw material can first, together with an aqueous solvent, be subjected to a coarse homogenization using big homogenizing blades and then a fine homogenization using small homogenizing blades. Based on the disclosure of the specification, persons having ordinary skills in the art can optionally change the homogenization process like the aforesaid arrangement. The arrangement of homogenization is not the critical technique in the present invention, so it is not described herein in detail.

In the method of the present invention, the high pressure homogenization in step (3) can be conducted at a pressure ranging from 100 bar to 2,000 bar. The high pressure homogenization can further homogenize the first extract and make the raw material to be extracted release the active ingredient(s), thereby obtaining a second extract with the active ingredient(s). The high pressure homogenization can be conducted at a temperature ranging from 25° C. to 85° C., to obtain a better efficiency of homogenization.

Before the high pressure homogenization in step (3), a sieving process can be optionally conducted to remove the bigger granules in the first extract, thereby, enhancing the efficiency of high pressure homogenization. The mesh size of the sieve used in the sieving process is not particularly limited. For example, sieves with 20 mesh to 400 mesh can be optionally used.

### EXAMPLE

The present invention will be further illustrated in detail with the following specific examples, wherein the adopted measurement apparatuses and methods are respectively described as follows:

1. Measurement Apparatus:
(1) UV/VIS spectrophotometer: purchased from Jasco company, product no.: V-360; and
(2) pH meter: purchased from EUTECH company, product no.: pH510.

2. Measurement Method

2.1 Determination of Betalain Amount

The determination method comprises the following steps:
1. Preparation of solutions:
   (1) Na₂HPO₄ solution (200 mM): Taking 1.4196 g of Na₂HPO₄ and adding deionized water until the total volume is 100 mL.
   (2) Citrate acid solution (200 mM): Taking 1.9213 g of citrate acid and adding deionized water until the total volume is 100 mL.
   (3) Mcllvaine's buffer: Taking 53.6 mL of Na₂HPO₄ solution (200 mM) and adding 46.4 mL of citrate acid (200 mM), then adjusting the pH value to 5.2.
2. Pre-treatment of sample
   (1) Liquid sample: Properly diluting the sample with Mcllvaine's buffer based on the concentration of sample (controlling the absorbance at 480 nm within 1).
   (2) Solid sample: Taking 10 g of the sample and adding Mcllvaine's buffer until the total volume is 100 mL, then agitating by an ultrasonic water bath until completely dissolving and properly diluting the sample with McIlvaine's buffer based on the concentration of sample (controlling the absorbance of 480 nm within 1).
3. Centrifuging the sample subjected to the above pre-treatment (3500 rpm, 5 minutes), and filtering by a filter paper no. 1 to obtain a filtrate.
4. Taking the filtrate into a 1-cm quartz tube and measuring its absorbance at 480 nm (i.e., OD₄₈₀).
5. Using the following formula to calculate the amount of betalain:

Betalain (μg/g)=[(OD₄₈₀×1%)/1120]×DF×(10⁶)
wherein “1120” is the absorbance of 1% betalain, and “DF” is the dilution factor of the sample at the time of the pre-treatment.

2.2. Determination of Betaxanthin Amount

The same determination method applied for the betalain above is used, but the absorbance for the sample solution at 538 nm (i.e., OD₅₃₈) is measured.

Using the following formula to calculate the amount of betaxanthin:

Betaxanthin (μg/g)=[(OD₅₃₈33 1%)/750]×DF×(10⁶)

In the formula, “750” is the absorbance of 1% betaxanthin, and “DF” is the dilution factor of the sample at the time of the pre-treatment.

2.3. Determination of Total Polyphenol Amount

1 g of homogenized sample is mixed with deionized water at a total volume of 10 mL, and agitated and extracted by an ultrasonic water bath for 30 minutes. Then, the mixture thus obtained is subjected to centrifugation (3,500 rpm, 5 minutes). The supernatant provided by the centrifugation is diluted until the absorbance at 750 nm (i.e., OD750) is smaller than 1 and thus is taken as a sample solution.

100 μL of sample solution is placed into a 15-mL centrifugation tube and 0.5 mL of Folin's phenol reagent is added therein. The sample solution and the Folin's phenol reagent are uniformly mixed and the resulting mixture is left to stand at room temperature for 3 minutes. Then, 0.4 mL of 7.5% sodium carbonate is added and uniformly mixed, and then is left to stand at room temperature for 30 minutes to obtain a mixture.

A 1 mL sample of the mixture is placed into a 1-cm quartz tube and its OD₇₅₀ is measured. Then, the measured absorbance is compared with a standard sample of total polyphenol, whose total polyphenol concentration is known, and the total polyphenol amount in the sample is calculated.

2.4. Determination of Total Flavonoid Amount

1 g of homogenized sample is mixed with deionized water at a total volume of 10 mL, and agitated and extracted by an ultrasonic water bath for 30 minutes. Then, the mixture thus obtained is subjected to centrifugation (3,500 rpm, 5 minutes). The supernatant provided by the centrifugation is diluted until the absorbance of 500 nm (i.e., OD₅₀₀) is smaller than 1 and thus is taken as a sample solution.

200 μL of sample solution is placed into a 15-mL centrifugation tube and deionized water is added until the total volume is 1.2 mL. 200 μL of 5% sodium nitrate is added therein and mixed uniformly, and then is left to stand for 6 minutes. Thereafter, 200 μL of aluminum nitrate is added and mixed uniformly, and then is left to stand for 6 minutes. 2 mL of 4% sodium hydroxide is further added and mixed uniformly. Then, 1.4 mL of deionized water is added and mixed uniformly to obtain a mixture. 200 μL of the mixture is placed into a 96-well plate and its OD₅₀₀ is measured. The measured absorbance is compared with a standard sample of total flavonoid, whose total flavonoid concentration is known. The total flavonoid amount in the sample is calculated.

2.5. Determination of Total Anthocyanidin

Two homogenized samples with the same volume are respectively diluted by using a 0.025 M potassium chloride buffer (pH 1.0) and a 0.4 M sodium acetate buffer (pH 4.5) to the extent that the absorbance thereof can be measured by a machine. The diluted sample solutions of potassium chloride and sodium acetate, respectively, are left to stand at room temperature for 15 minutes.

The sample solutions of potassium chloride and sodium acetate are respectively measured for their absorbance at 510 nm and 700 nm (i.e., OD₅₁₀ and OD₇₀₀). Then, the total anthocyanidin amount is calculated by the following formula:

Amount (mg/g)=((A*molecular weight*dilution factor)/molar absorptivity)*1

In the formula, A refers to a value obtained by subtracting “the difference of OD₅₁₀ and OD₇₀₀ in the sample solutions of sodium acetate” from “the difference of OD₅₁₀ and OD₇₀₀ in the sample solutions of potassium chloride”, the molecular weight is 449.2 g/mol, and the molar absorptivity is 26,900 1/mol*cm.

2.6. Determination of Serotonin Amount

The sample is subjected to a pre-treatment as follows:
1. Solid sample:
   (1) Taking 1 g of the sample and placing into a centrifugation tube, and adding 50 mL of deionized water and agitating by an ultrasound for 5 minutes to obtain a mixture;
   (2) Placing the mixture into a new 15-mL centrifugation tube and conducting centrifugation (4,000 rpm, 10 minutes), and filtering to obtain a filtrate.
2. Liquid sample:
   (1) Taking 1 mL of sample and adding deionized water until the total volume is 25 mL to obtain a mixture; and
   (2) Placing the mixture into a new 15-mL centrifugation tube and conducting centrifugation (4,000 rpm, 10 minutes), and filtering to obtain a filtrate.

The filtrate obtained from the above pre-treatment is analyzed by a liquid chromatography-mass spectrometry under the following conditions to determine the amount of serotonin in sample:
1. Chromatography column (purchased from Waters company, product no.: ACQUITY UPLC HSS T3, 2.1×100 mm to 1.8 gm);
2. Temperature of chromatography column: 30° C.;
3. Solution of mobile phase: aqueous solution of 0.1% formic acid: acetonitrile solution with 0.1% formic acid=90: 10;
4. Flow rate: 0.3 mL/min;
5. Analysis time: 3 minutes;
6. Volume of input: 1 μL;
7. Capillary voltage: Electrospray ionization cation (ESI+) adopts 2.5 kV;
8. Ion source temperature: 150° C.;
9. Desolvation temperature: 350° C.;
10. Desolvation flow: 800 L/hr; and
11. Monitoring mode: multiple reaction monitoring, monitoring ion pairs, cone voltage and collision energy are shown as follows.

| | | Ion pairs | cone | collision |
|---|---|---|---|---|
| | Ionization | precursor ion (m/z) > | voltage | energy |
| Analyte | mode | product ion (m/z) | (V) | (eV) |
| Serotonin | ESI+ | 176.6488 > 160* | 20 | 40 |
| | | 176.6488 > 115 | 20 | 30 |
| | | 176.6488 > 132 | 20 | 18 |

*quantitative ion pairs

3. Extraction Experiment

3.1. Extraction of Active Ingredients in Chenopodium formosanum

### Example 1

Shelled Chenopodium formosanum was subjected to a breaking process by a crusher for 30 seconds to obtain a powder of Chenopodium formosanum. The powder of Chenopodium formosanum was uniformly mixed with water (powder of Chenopodium formosanum: water 32 1:1-10, at a volume ratio). The mixture thus obtained was placed into a homogenizer. At a normal temperature or 85° C., a primary homogenization was conducted by using blades for coarse homogenization for 2 to 10 minutes. Then, after changing the blades to ones for fine homogenization, further homogenization was conducted for 2 to 10 minutes to obtain a crude extract (first extract).

The crude extract was subjected to a sieving process by a 400 mesh sieve to obtain a filtrate. Then, the filtrate was placed into a high pressure homogenizer and subjected to a high pressure homogenization. The high pressure homogenization was conducted at a temperature ranging from 25° C. to 85° C. and a pressure ranging from 200 bar to 800 bar for 1 to 2 minutes, to obtain an extract of Chenopodium formosanum (second extract).

The amounts of active ingredients such as betalain, betaxanthin, total polyphenol and total flavonoid in the extract of Chenopodium formosanum were determined by the aforesaid determination methods. The results are shown in Table 1.

### Comparative Example 1

Shelled Chenopodium formosanum was grinded and the grinded Chenopodium formosanum was sieved (sieve: 10 mesh) to provide a powder of Chenopodium formosanum. The powder of Chenopodium formosanum was mixed with water (powder of Chenopodium formosanum: water=1:10, at a volume ratio), and then was extracted at 75° C. for 0.5 hour to provide a crude extract.

The crude extract was cooled to 25° C. and subjected to a centrifugation at a rotary speed of 5000 rpm for 10 minutes. The supematant thus obtained was filtered by a 400 mesh filter to obtain a filtrate. At 60° C., the filtrate was subjected to vacuum concentration to obtain a concentrated extract.

The amounts of active ingredients such as betalain, betaxanthin, total polyphenol and total flavonoid in the concentrated extract were determined by the aforesaid determination methods. The results are shown in Table 1.

TABLE 1
Extraction results of active ingredients in Chenopodium formosanum

| | Homogenization | | | Active ingredient | | | |
|---|---|---|---|---|---|---|---|
| | temperature of | Sugar | | | | Total | Total |
| | homogenizer | degree | pH | betalain | betaxanthin | polyphenol | flavonoid |
| | (° C.) | (Brix) | value | (μg/g) | (μg/g) | (ppm) | (ppm) |
| Comparative | — | 1.865 | 5.74 | 12.7 | 14.3 | 147.5 | 666.9 |
| example 1 | | | | | | | |
| Example 1 | Normal | 1.860 | 6.30 | 720.0 | 512.1 | 1161.5 | 2136.7 |
| | temperature | | | | | | |
| | 85 | 1.960 | 6.17 | 738.0 | 525.9 | 839.7 | 2174.7 |

As shown in Table 1, whether the temperature was at normal or at 85° C., the extraction efficiencies of extraction method of the present invention for the active ingredients in Chenopodium formosanum are both better than that of conventional method, wherein the extraction efficiencies of extraction method of the present invention for betalain is particularly 60-fold more than that of the conventional method.

3.2. Extraction of Active Ingredient in Black Tomato

### Example 2

Black tomato was subjected to a breaking process by a crusher for 30 seconds to obtain fragments of the black tomato. The fragments of the black tomato were uniformly mixed with water (fragments of black tomato: water=1:2-10, at a volume ratio). The mixture thus obtained was placed into a homogenizer. At 40° C., a primary homogenization was conducted by using blades for coarse homogenization for 2 to 10 minutes. Then, after changing the blades to ones for fine homogenization, further homogenization was conducted for 2 to 10 minutes to obtain a crude extract (first extract).

The crude extract was subjected to a sieving process by a sieve with 20 mesh to 100 mesh to obtain a filtrate. Then, the filtrate was placed into a high pressure homogenizer and subjected to a high pressure homogenization. The high pressure homogenization was conducted at a temperature ranging from 25° C. to 45° C. and a pressure ranging from 200 bar to 600 bar for 1 to 2 minutes to obtain an extract of black tomato (second extract).

The amount of active ingredient such as total anthocyanidin in the extract of black tomato was determined by the aforesaid determination methods. The results are shown in Table 2.

### Comparative Example 2

The black tomato was subjected to a breaking process by a crusher to obtain fragments of the black tomato. The fragments of black tomato were uniformly mixed with water and subjected to centrifugation. The supernatant thus obtained was filtered by a filter to obtain a filtrate. At 60° C., the filtrate was subjected to vacuum concentration to provide a concentrated extract.

The amount of active ingredients such as total anthocyanidin in the concentrated extract of black tomato was determined by the aforesaid determination methods. The results are shown in Table 2.

TABLE 2
Extraction results of active ingredients in black tomato

| | | | Total anthocyanidin |
|---|---|---|---|
| | Sugar degree | pH value | (μg/g) |
| Comparative | 1.935 | 3.42 | 0.95 |
| example 2 | | | |
| Example 2 | 2.135 | 3.22 | 1.55 |

As shown in Table 2, the extraction efficiencies of extraction method of the present invention for the active ingredients in black tomato are better than that of the conventional method.

3.3. Extraction of Active Ingredient in Banana Peel

### Example 3

A banana peel was subjected to a breaking process by a crusher for 30 seconds to obtain fragments of the banana peel. The fragments of banana peel were uniformly mixed with water (fragments of banana peel: water=1:2-10, at a volume ratio). The mixture thus obtained was placed into a homogenizer. At 85° C., a primary homogenization was conducted by using blades for coarse homogenization for 2 to 10 minutes. Then, after changing the blades to ones for fine homogenization, further homogenization was conducted for 2 to 10 minutes to obtain a crude extract (first extract).

The crude extract was subjected to a sieving process by a sieve with 20 mesh to 100 mesh to obtain a filtrate. Then, the filtrate was placed into a high pressure homogenizer and subjected to a high pressure homogenization. The high pressure homogenization was conducted at a pressure ranging from 200 bar to 600 bar to obtain an extract of the black tomato (second extract).

The amount of active ingredient such as serotonin in the extract of banana peel was determined by the aforesaid determination methods. The results are shown in Table 3.

### Comparative Example 3

A banana peel was subjected to a breaking process by a crusher for 30 seconds to obtain the fragments of a banana peel. The fragments of the banana peel were uniformly mixed with water (fragments of banana peel: water=1:6 at volume ratio) to provide a crude extract.

The crude extract was cooled to 25° C. and subjected to a centrifugation at a rotary speed of 5000 rpm for 10 minutes. The supernatant thus obtained was filtered by a 400 mesh filter to obtain a filtrate. At 60° C., the filtrate was subjected to vacuum-concentration to provide a concentrated extract. The concentrated extract was subjected to a freeze drying to obtain a dried extract of the banana peel.

The amount of active ingredient such as serotonin in the extract of banana peel was determined by the aforesaid determination methods. The results are shown in Table 3.

TABLE 3
Extraction results of active ingredients in banana peel

| | | pH | Serotonin |
|---|---|---|---|
| | Sugar degree | value | (μg/g) |
| Comparative | 0.805 | 2.79 | 0.36 |
| example 3 | | | |
| Example 3 | 1.540 | 2.97 | 0.37 |

As shown in Table 3, the extraction efficiencies of extraction method of the present invention for the active ingredient in banana peel are better than that of the conventional method.

The above results indicate that no matter what active ingredient was extracted from Chenopodium formosanum, black tomato or banana peel, the extraction efficiencies of extraction method of the present invention is comprehensively better than that of conventional methods. That is, the extraction method of the present invention can be applied generally and the efficiency is excellent.

The above Example is used to illustrate the principle and efficacy of the present invention and show the inventive features thereof. People skilled in this field may proceed with a variety of modifications and replacements based on the disclosures and suggestions of the invention as described without departing from the principle and spirit thereof. Therefore, the scope of protection of the present invention is that as defined in the claims as appended.

### BRIEF DESCRIPTION OF NUMERAL REFERENCES

None.

What is claimed is:

1. A method for extracting an active ingredient from a raw material to be extracted, comprising the following steps:
(1) mixing the raw material to be extracted with an aqueous solvent to provide a mixture;
(2) conducting a homogenization of the mixture to obtain a first extract with the active ingredient; and
(3) conducting a high pressure homogenization of the first extract to obtain a second extract with the active ingredient,
wherein the homogenization in step (2) is conducted at a temperature ranging from normal temperature to 100° C.

2. The method as claimed in claim 1, wherein the high pressure homogenization in step (3) is conducted at a pressure ranging from 100 bar to 2,000 bar.

3. The method as claimed in claim 1, wherein the homogenization in step (2) is conducted at a temperature ranging from normal temperature to 90° C.

4. The method as claimed in claim 1, wherein the raw material to be extracted is selected from the group consisting of Chenopodium formosanum, black tomato, banana peel and combinations thereof.

5. The method as claimed in claim 4, wherein the raw material to be extracted is Chenopodium formosanum, and the active ingredient is selected from the group consisting of betalain, betaxanthin, polyphenol, flavonoid and combinations thereof.

6. The method as claimed in claim 5, wherein the homogenization in step (2) is conducted at a normal temperature or a temperature ranging from 80° C. to 90° C.

7. The method as claimed in claim 4, wherein the raw material to be extracted is a black tomato, and the active ingredient is anthocyanidin.

8. The method as claimed in claim 7, wherein the homogenization in step (2) is conducted at a temperature ranging from 35° C. to 45° C.

9. The method as claimed in claim 4, wherein the raw material to be extracted is a banana peel, and the active ingredient is serotonin.

10. The method as claimed in claim 9, wherein the homogenization in step (2) is conducted at a temperature ranging from 80° C. to 90° C.

11. The method as claimed in claim 1, which further comprises, before step (1), breaking the raw material to be extracted.

12. The method as claimed in claim 1, which further comprises, before step (3), sieving the homogenate by using a sieve with 20 mesh to 400 mesh.

13. The method as claimed in claim 1, wherein the aqueous solvent is water.
